(19) United States
(12) Patent Application Publication
(10) Pub. No.: US 2026/0054283 A1
(43) Pub. Date: Feb. 26, 2026

(54) A Dispensing Head

(71) Applicant: Reckitt & Colman (Overseas) Hygiene Home Limited, Slough, Berkshire (GB)

(72) Inventors: Alberto BUOGO, Hull (GB); Guillaume KENSICHER, Hull (GB); Christopher Brian WITTY, Hull (GB)

(21) Appl. No.: 19/105,578

(22) PCT Filed: Aug. 31, 2023
(86) PCT No.: PCT/EP2023/073900

§ 371 Date: Feb. 21, 2025

(30) Foreign Application Priority Data
Aug. 31, 2022 (GB) 2212593.4

(51) Int. Cl. B05B 15/68; A45D 34/00

(52) U.S. Cl. CPC B05B 15/68; A45D 2034/005

(57) ABSTRACT

A refill for a liquid fragrance dispensing device, the refill configured to be inserted into the fragrance dispensing device; wherein the refill comprises a rotatable spray head and a bottle, the rotatable spray head further comprising a nozzle; the spray head is configured to dispense liquid fragrance from the bottle; wherein the spray head is configured to be rotated by the dispensing device by contacting at least one jaw of the dispensing device as the bottle is inserted into the dispensing device; and wherein the spray head further comprises at least one vertical rib and the at least one rib is configured to contact the at least one jaw to align the spray head in the dispensing device.

Air fresheners may be dispersed into a room using a dispenser. In general, the dispenser has a spray head that allows the fluid to be directed into a room. The spray head is activated to release the fluid in a particular direction in the room.

When a dispensing device is used in conjunction with a fragrance to spray a fluid into the room the direction of the spray may be important to the performance of the device. A misaligned spray head may be associated with reduced performance. For example, when the spray head is misaligned the fluid may be dispensed in an undesired direction or the fluid may be sprayed, at least partially, on to the housing of the dispenser.

The present invention relates to a device for spraying a fluid and particularly, but not exclusively to a device for spraying fluids such as fragrances, deodorizing fluids and/or a pest control material or the like. The present invention also relates to a method of using such a device.

In a first aspect of the invention, there is a refill for a liquid fragrance dispensing device, the refill configured to be inserted into the fragrance dispensing device; wherein the refill comprises a rotatable spray head and a bottle, the rotatable spray head further comprising a nozzle; the spray head is configured to dispense liquid fragrance from the bottle; wherein the spray head is configured to be rotated by the dispensing device by contacting at least one jaw of the dispensing device as the bottle is inserted into the dispensing device; and wherein the spray head further comprises at least one vertical rib and the at least one vertical rib is configured to contact the at least one jaw to align the spray head in the dispensing device.

In a further aspect, there is a rotatable spray head for a refill configured to be inserted into the fragrance dispensing device; the rotatable spray head is attachable to the refill; the rotatable spray head comprises a nozzle and the spray head is configured to dispense liquid fragrance from a refill via the nozzle; wherein the spray head is configured to be rotated by the dispensing device by contacting at least one jaw of the dispensing device as the bottle is inserted into the dispensing device; and wherein the spray head further comprises at least one vertical rib and the at least one vertical rib is configured to contact the at least one jaw to align the spray head in the dispensing device.

In a further aspect, there is liquid fragrance dispensing device comprising a refill, the refill configured to be inserted into the fragrance dispensing device; wherein the refill comprises a rotatable spray head and a bottle, the rotatable spray head further comprising a nozzle; the spray head is configured to dispense liquid fragrance from the bottle; wherein the spray head is configured to be rotated by the dispensing device by contacting at least one jaw of the dispensing device as the bottle is inserted into the dispensing device; and wherein the spray head further comprises at least one vertical rib and the at least one vertical rib is configured to contact the at least one jaw to align the spray head in the dispensing device.

Aspects of the disclosure are also described in detail, by way of example only, with reference to the accompanying drawings, in which:

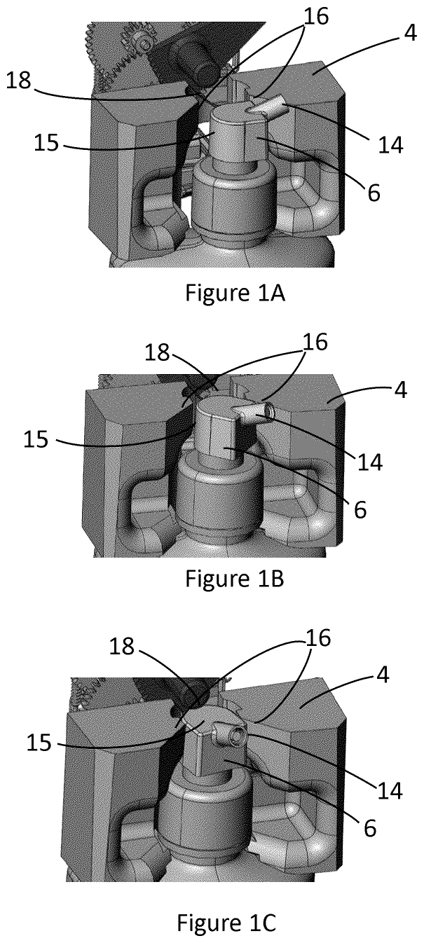

FIG. 1A shows a spray head of a refill in a first position during installation into a dispensing device;

FIG. 1B shows the spray head of the refill in a second position during installation into the dispensing device;

FIG. 1C shows the spray head of the refill in a third position during installation into the dispensing device.

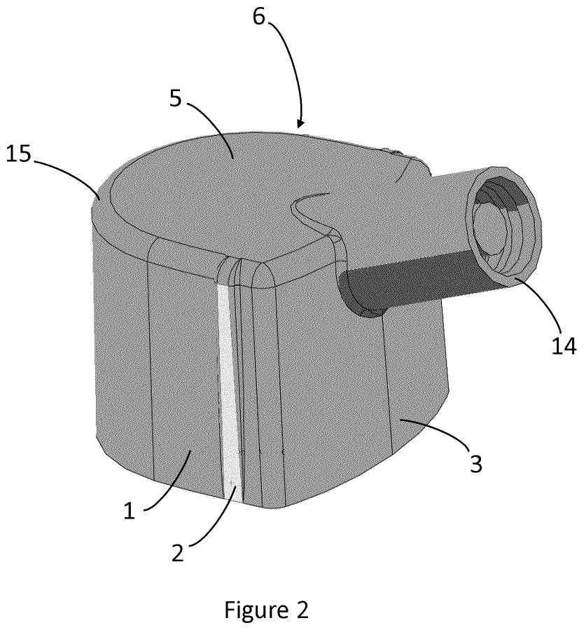

FIG. 2 shows the spray head of the refill with a locating rib;

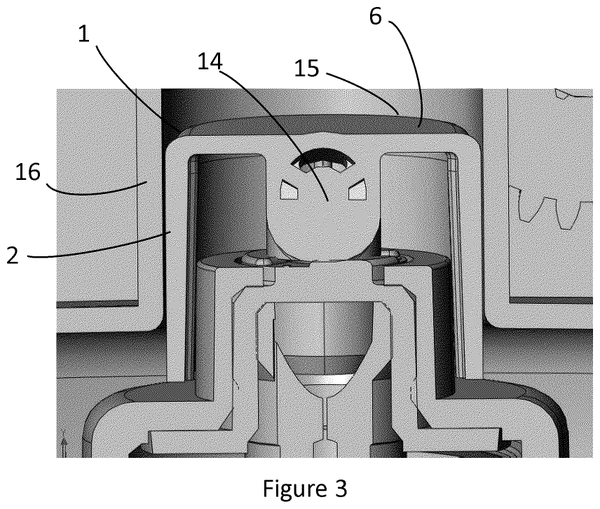

FIG. 3 shows the spray head of FIG. 2 located in the dispensing device.

Embodiments will now be described with reference to the Figures.

FIGS. 1A, 1B and 1C show a spray head 6 for a refill. The figures show an illustration of the refill being placed into a dispensing device 4. As illustrated, the spray head 6 rotates to the desired orientation of the spray head. The spray head 6 is configured to be rotatable, for example the spray head 6 is configured to be attached to refill so that it rotatable about the refill.

The spray head 6 comprises a rounded part 15 and a nozzle 14 for dispensing the fluid. In the example illustrated, the rounded part 15 is located at the opposite side of the spray head 6 relative to the nozzle 14. The dispensing device 4 comprises a mouth having a pair of jaws 16 and a spray head receiving portion 18.

As illustrated in FIGS. 1A, 1B and 1C the jaws 16 are configured to guide the spray head 6 so that the nozzle 14 is oriented with the nozzle 14 in the desire location. For example, the nozzle 14 of spray head 6 is rotated by the jaws 16 to be directed towards the spray orifice when docked in the spray head receiving portion 18. The jaws 16 cooperate with the rounded part 15 of the spray head 6 so that the nozzle 14 is in the correct orientation. For example, if the spray head 6 is incorrectly oriented one of the jaws 16 will contact the rounded part 15 so that a rotational force is imparted on the spray head 6. The rotational force leads to the spray head 6 rotating so that the nozzle 14 is correctly oriented with the nozzle 14 directed towards the spray orifice when docked in the spray head receiving portion 18.

FIG. 1A shows the spray head 6 entering the jaws 16 with the nozzle 14 oriented in an undesired direction. FIG. 1B shows the spray head 6 coming into contact with one of the jaws 16. The contact between the jaw 16 and the spray head 6 rotates the spray head 6 towards the desired orientation. FIG. 1C shows the spray head 6 docked in the spray head 6 receiving portion 18 with the spray head correctly oriented with the nozzle 14 directed towards the spray orifice.

The spray head 6 is shaped to contact the jaws 16 if the spray head 6 is in an undesired orientation. In the example illustrated, the rounded part 15 of the spray head 6 comprises a semi-circular portion with straight portions on either side of the semi-circular portion and the nozzle 14 at the side of the spray head opposite to the semi-circular portion, for example a “D” shape, and the jaws 16 are symmetrical to one another and comprise a straight angled portion. As illustrated in FIGS. 1A, 1B and 1C, the shape of the spray head 6 and the jaws 16 cooperate so that one side of the spray head 6 contacts one of the jaws 16 to rotate the spray head so that the spray head 6 is moved into a desired orientation e.g. an orientation where the spray head is oriented towards the spray orifice.

FIG. 2 shows the spray head 6 illustrated in FIGS. 1A-1C in more detail. The spray head 6 shown in FIG. 2 comprises a rounded part 15, a side part 1, a front part 3, a rib 2, a top part 5 and a nozzle 14. The spray head 6 is configured with the nozzle 14 at least partially extending from the front part 3 of the spray head 6. The rounded part 15 is connected to the front part 3 via a pair of side parts 1. The rib 2 is located on the side part 1. In the example illustrated, the spray head 6 comprises a pair of ribs with each rib 2 located on each of the pair of side parts 1. In the example illustrated the rib is located in a vertical orientation.

FIG. 3 is an illustration of a cross section of the spray head 6 located in the dispensing device 4. The spray head 6 is located with the rib 2 in contact with the jaw 16 to assist the alignment of the nozzle 14 of the spray head 6 in the dispensing device 4.

The rib 2 is configured so that it extends from the side part 1. The rib 2 is configured to contact the jaw 16 when the spray head 6 is located in the dispensing device 4. The contact between the jaw 16 and the rib 2 assists with the alignment of the nozzle. For example, the contact between the rib 2 and the jaw 16 reduces the degree of movement of the spray head 6 in the pair of jaws 16. The contact between the rib 2 of the spray head 6 and the jaw 16 reduces the rotational freedom of the spray head and therefore improves the alignment of the spray head 6.

The optimum alignment of the nozzle 14 relative to the device improves the performance by reducing the amount of liquid being dispensed in an undesired direction. For example, the misalignment of the nozzle may lead to liquid ejected from the nozzle being sprayed on to parts of the dispensing device leading to both undesired liquid on the dispenser and a reduction in the range of the spray.

The size of the rib is selected to provide an optimal alignment of the nozzle 14 of the spray head 6 whilst limiting the friction between the spray head and the jaws 16. For example, the size of the rib 2 is selected to correctly align the nozzle 14 whilst keeping the friction between the spray head 6 and the jaw 16 at a level that allows the mechanism to reliably and efficiently operate the spray mechanism. For example, the size of the rib 2 is selected so that the friction between the rib 2 and the jaw 16 is sufficiently low to allow the motor to activate the spray whilst assisting with the alignment of the spray head.

In an example, the refill and dispensing mechanism is configured to dispense a non-aerosol liquid. Dispensing a non-aerosol liquid requires the spray head to be activated with sufficient velocity to project the liquid into the room with the required range. In this example, the rib 2 is selected so that the mechanism of the dispensing device can apply a sufficient velocity to allow the spray to be ejected with the sufficient range. The friction between the rib 2 and the jaw 16 may reduce the velocity of the spray head when activated by the motor and the rib size is selected to allow the motor to provide sufficient velocity to the spray head to dispense the liquid into the room with the required range.

In the example illustrated, the rib 2 has a width that is a fraction of the side portion 1. For example, the width of the rib is between 10% and 30% of the side portion, for example between 15% and 25%, for example 18% to 22%. In an example, the rib is 1.2 mm wide and the side portion is 5.8 mm wide.

In the example illustrated in FIG. 2, the rib 2 has a wedge shape. For example, one part of the rib projects from the side part 1 further than another part of the rib. In the example shown in FIG. 2 the wedge shape is such that the rib 2 projects further at the part of the rib proximate the top part 5 of the spray head 6 relative to the part of the rib furthest from the top part 5 of the spray head 6.

In the example illustrated, the rib 2 extends from the side portion at the maximum extension between 0.2% and 1% of the width of the spray head, for example between 0.4% and 0.8%, for example 0.5% to 0.7%, for example 0.6%.

In the example illustrated the wedge shape of the rib 2 forms an angle of between 1 to 2% with the side portion of the spray head, for example 1.2% to 1.8%, for example 1.3% to 1.5%. In a further example the extension of the rib from the side of the spray head is constant i.e. the rib extension from the side of the spray head is uniform.

In the examples above, the spray head is moulded with the ribs 2 extending from the side part 1. In other examples, the spray head may comprise ribs that are formed separately and attached to the side part 1.

In the examples described, the dispensing device dispenses a liquid fragrance. The dispensing device may also dispense deodorizing fluids and/or a pest control material or the like.

Further modifications and developments can be made without departing from the scope of the invention described herein.

1. A refill for a liquid fragrance dispensing device, the refill configured to be inserted into the fragrance dispensing device;
wherein the refill comprises a rotatable spray head and a bottle, the rotatable spray head further comprising a nozzle;
the spray head is configured to dispense liquid fragrance from the bottle;
wherein the spray head is configured to be rotated by the dispensing device by contacting at least one jaw of the dispensing device as the bottle is inserted into the dispensing device; and
wherein the spray head further comprises at least one vertical rib and the at least one rib is configured to contact the at least one jaw to align the spray head in the dispensing device.

2. The refill according to claim 1, wherein the spray head further comprises a rounded part, a pair of side parts, a front part and a top part;
wherein the nozzle at least partially extends from the front part of the spray head and the rounded part is connected to the front part via a pair of side parts; and
wherein the at least one rib is located on at least one of the pair of side parts.

3. The refill according to claim 2, wherein the rib has a wedge shape with one part of the rib projecting from a side part further than another part of the rib.

4. The refill according to claim 2, wherein the width of the rib is between 10% and 30% of the side portion.

5. The refill according to claim 2, wherein the rib extends from the side portion at the maximum extension between 0.2% and 1% of the width of the spray head.

6. The refill according to claim 3, wherein the wedge shape of the rib forms an angle of between 1° to 2°.

7. A rotatable spray head for a refill configured to be inserted into a fragrance dispensing device; wherein
the rotatable spray head is attachable to the refill;
the rotatable spray head comprises a nozzle and the spray head is configured to dispense liquid fragrance from a refill via the nozzle;
wherein the spray head is configured to be rotated by the dispensing device by contacting at least one jaw of the dispensing device as the bottle is inserted into the dispensing device; and
wherein the spray head further comprises at least one vertical rib and the at least one rib is configured to contact the at least one jaw to align the spray head in the dispensing device.

8. A liquid fragrance dispensing device comprising a refill according to claim 1.

9. The refill according to claim 2, wherein the rib extends from the side portion at the maximum extension between 0.4% and 0.8%.

10. The refill according to claim 2, wherein the rib extends from the side portion at the maximum extension between e 0.5% to 0.7%.

11. The refill according to claim 2, wherein the rib extends from the side portion at the maximum extension of 0.6%.

12. The refill according to any of claims 3, wherein the wedge shape of the rib forms an angle of between 1.2° to 1.8° with the side portion of the spray head.

13. The refill according to any of claims 3, wherein the wedge shape of the rib forms an angle of between 1.3° to 1.5° with the side portion of the spray head.

14. The refill according to claim 2, wherein the width of the rib is between 15% and 25% of the side portion.

15. The refill according to claim 2, wherein the width of the rib is between 18% to 22% of the side portion.
